(19) United States
(12) Patent Application Publication
(10) Pub. No.: US 2019/0255088 A1
(43) Pub. Date: Aug. 22, 2019

(54) BIOCOMPATIBLE ORGANO-INORGANIC NANOCOMPOSITES

(71) Applicant: The Board of Regents of the University of Oklahoma, Norman, OK (US)

(72) Inventors: Priyabrata Mukherjee, Edmond, OK (US); Md. Nazir Hossen, Oklahoma City, OK (US); Resham Bhattacharya, Edmond, OK (US)

(21) Appl. No.: 16/277,475

(22) Filed: Feb. 15, 2019

Related U.S. Application Data
(60) Provisional application No. 62/631,752, filed on Feb. 17, 2018.

(51) Int. Cl. A61K 31/7088; A61P 35/00; A61K 47/69; A61K 47/02

(52) U.S. Cl. CPC A61K 31/7088; A61K 47/02; A61K 47/6911; A61P 35/00

(57) ABSTRACT

Disclosed is an organo-inorganic nanocomposite (OINC) and a method of use thereof, the OINC containing a lipid membrane component made of a cationic lipid, a fusogenic co-lipid, and a pore forming surfactant; and a cargo-inorganic conjugate component made of a negatively-charged cargo molecule bound electrostatically or covalently to a negatively-charged biocompatible inorganic nanoparticle wherein the cargo-inorganic component is substantially encapsulated within the lipid membrane component forming the OINC.

### CROSS-REFERENCE TO RELATED APPLICATIONS

The present application claims priority to U.S. Provisional Application Ser. No. 62/631,752, filed Feb. 17, 2018, which claims the benefit under 35 U.S.C. 119(e), the disclosure of which is hereby expressly incorporated herein by reference.

### TECHNICAL FIELD

The present disclosure relates generally to nanocomposites and more particularly, but not by way of limitation, to compositions and methods for biocompatible organo-inorganic nanocomposites.

### BACKGROUND

For decades, therapeutic molecules (e.g. siRNA, miRNA, peptides, proteins and small molecules) have been utilized as tools to treat diseases, including cancer. However, the effectiveness of therapeutic molecules (TMs) has been posed a problem due to the lack of proper delivery system for these molecules into specific cells and tissues. The major concerns include the low cell penetration, pleotropic activities of TMs and the susceptibility of degradation by blood enzymes and cells, resulting in the low availability of the TMs at or into the disease site. Therefore, it is clear that the ability to deliver adequate therapeutic amounts of TMs and extending the time of exposure, would substantially boost the effectiveness and efficacy of TMs. A delivery system that is targeted to the desired site of action will increase efficacy of TMs by minimizing dose levels, thereby decreasing toxicity, and reducing their nonspecific distribution into healthy tissues.

Liposomes are nanocomposites having a bilayer membrane structure made of amphiphilic lipid molecules and have long been used for drug delivery. During the past 25 years, the U. S. Food and Drug administration (USFDA) approved many liposomal products, including doxil (in 1995) for the treatment of AIDS-related Kaposi's sarcoma, breast cancer, ovarian cancer, and other solid tumors; daunoxome (in 1996) for advanced HIV-associated Kaposi's sarcoma and irinotecan (in 2015) for metastatic pancreatic cancer following gemcitabine-based therapy. Both liposomal lurtotecan and cisplatin containing targeted liposomes are now in phase II clinical trials for topotecan-resistant ovarian cancer and for advanced or refractory tumor respectively. Despite the advantageous features of liposomes as a delivery vehicle, the applications of liposomes have been limited by their instability due to the short circulation time in the circulation and its non-specific toxicities. An extensively used approach to stabilize liposomes is to coat their surface with a “stealth” material such as polyethylene glycol (PEG) for extending the residence time of the particles in the circulation. However, PEGylation leads to the poor cellular uptake of liposomes to target cells (known as the PEG dilemma). In addition, the attachment of targeting ligands using PEG spacer to the liposome may improve the efficacy of therapeutics with minimal toxicities. This also poses the therapeutic heterogeneity depending on the number of receptors expressed on the cell surface.

Small interfering RNA (siRNA) has been identified as a category of molecule that has therapeutic potential for causing sequence-specific gene knockdown in mammalian cells. However, a key challenge in realizing the full potential of siRNA is the efficient delivery of siRNA into cells because the physicochemical characteristics of siRNAs, including (1) high molecular weight, (2) anionic charge and (3) hydrophilicity pose obstacles to their passage across the plasma membrane of most cell types. For the effective delivery of siRNA, the surface charge of liposomes affects all three of these characteristics, including (i) a prolong blood circulation time, (ii) efficient penetration into tumor tissues, and (iii) high cellular uptake and efficient endosomal escape. Liposomes have been widely studied as carriers for the delivery of siRNA. As noted, on the basis of their lipid compositions, liposomes are mainly classified as cationic, anionic and neutral. Several liposomal siRNA delivery formulas, including ALN-VSPO2 (targeting KSP and VEGF genes), siRNA-EphA2-DOPC (targeting EphA2 gene), and Atu027 (targeting PKN3 gene), are now in clinical trials.

Several studies have reported that in the blood circulation, both cationic and anionic liposomes have a short circulation time due to their respective positive and negative surface charge, which increases their complement activation and macrophage uptake. Both types of liposomes also tend to have poor tumor penetration due to their low accumulation in tumor tissue. In addition, anionic liposomes, along with an inefficient endosomal escape can have a low cellular uptake in cancer cells due to repulsive force against anionic membranes. In comparison to both cationic and anionic liposomes, neutral liposomes are relatively stable and longer blood circulation time. The tumor penetration of neutral liposomes is also more efficient than that of both cationic and anionic liposomes. However, despite the improvements and advances noted above, there is an unmet medical need for developing more effective lipid-based nanocomposite formulations for the delivery of these therapeutic molecules. It is to satisfying this unmet need that the present disclosure is directed.

### BRIEF DESCRIPTION OF THE DRAWINGS

Several embodiments of the present disclosure are hereby illustrated in the appended drawings. It is to be noted however, that the appended drawings only illustrate several typical embodiments and are therefore not intended to be considered limiting of the scope of the inventive concepts disclosed herein. The figures are not necessarily to scale and certain features and certain views of the figures may be shown as exaggerated in scale or in schematic in the interest of clarity and conciseness. The patent or application file contains at least one drawing executed in color. Copies of this patent or patent application publication with color drawing(s) will be provided by the Office upon request and payment of the necessary fee.

FIG. 1 shows chemical structures of certain lipids used in formulations of the present disclosure.

FIG. 2 shows the size and zeta potential of several cystathione-β-synthatase (CBS) siRNA-loaded organo-nanocomposite (ONC) formulations screened for use in the presently described work. The sizes and zeta of ONCs denoted in Table 1 as F1, F2, F3, F4, F5, F6, F7, F8, F9, F10 and E-LP (empty-ONC, without siRNA) were measured by dynamic light scatter microscopy (DLS) (n=3).

FIG. 3 shows percent entrapment of the CBS siRNA into the ONCs as measured using ribogreen assay (n=3).

FIG. 4 represents optimization of the CBS siRNA dose. CP20 cells (2×10⁵) were transfected with CBS siRNA at several doses of 0, 10, 25, 50 and 133 nM or control siRNA (CTL) at a dose of 133 nM using hiperfect transfection reagent in complete 10% FBS media and post 48 h the extent of CBS silencing was determined at the protein level by western blot. GAPDH was used as a loading control. Quantitative measurement of CBS protein expression was analyzed by Image J and has been shown as % silencing activity of CBS protein compared to that of control siRNA, normalized by GAPDH (n=3).

FIG. 5 shows a comparison of the ONCs on the basis of their CBS silencing activity. CP20 cells were treated with CBS siRNA-ONCs (F1-F10) at a dose of 25 nM or control (CTL) siRNA-ONCs at the same dose at same conditions. The extent of CBS silencing at protein level was determined by western blot. GAPDH was used as loading control. Quantitative measurement of CBS protein expression is shown (n=3) in the bar diagram.

FIG. 6 is a schematic illustration showing of an example of an siRNA-loaded organo-inorganic nanocomposite (siRNA-OINC) constructed in accordance with the present invention with DOTAP and DOPE as structural lipid components, Tween 20 as a pore-forming surfactant, and an electrostatic or covalent conjugate comprising gold nanoparticles (inorganic nanoparticle), and an siRNA as the cargo molecule in an aqueous core.

FIG. 7 characterizes CBS siRNA organo-nanocomposites (ONCs) and OINCs according to size (a), zeta potential (b), and percent entrapment of the siRNA (c). The size and zeta potential were measured by dynamic light scatter microscopy (DLS) and entrapment was measured using the ribogreen assay (n=3).

FIG. 8 examines CBS silencing by various OINC formulations. Optimization of ratios in inorganic nanoparticles (INPs) incorporation into CBS siRNA-ONCs. CP20 cells (2×10⁵) were treated with either CBS siRNA conjugated with at various ratios of INPs, 1:5, 1:10 and 1:20 w/w, or without CBS siRNA via ONCs with INPs at a final concentration of 25 nM, or empty-ONCs or remains untreated in complete 10% FBS media and post 48 h the extent of CBS silencing was determined at the protein level by western blot. GAPDH was used as loading control.

FIG. 9 shows results using CP20 cells (2×10⁵) were either treated with control siRNA-ONCs, OINCs, INPs, CBS siRNA-OINCs, a mixture (INPs+ CBS siRNA-ONCs), or pretreated with INPs for 2 h, following a treatment with CBS siRNA-ONCs at a dose of 25 nM at the same conditions. The extent of CBS silencing was determined at the protein level by western blot. GAPDH was used as a loading control. Results show that incorporation of INPs at a ratio of 1:10 forming OINCs results in better silencing activity.

FIG. 10 shows a quantitative measurement CBS protein expression using the results of FIG. 9 analyzed by Image J, percent silencing activity of CBS protein is compared to that of control siRNA, normalized by GAPDH (n=3).

FIG. 11 shows serum stability results of free CBS siRNA, CBS siRNA-ONCs, CBS siRNA-OINCs, complex (Hiperfect+CBS siRNA) and conjugate (INPs+CBS siRNA) (1 ug each) incubated with 100% FBS (1:1 v/v) at 37° C. for 15 mins, 24 h, 48 h and 72 h. 1.5% agarose gel electrophoresis was performed in the presence of TBE buffer.

FIG. 12 shows time-dependent cellular uptake of fluorescence labeled control (CTL) siRNA-OINCs by CP20 cells. Cells were treated with cy5 siRNA, cy5 siRNA-ONCs, cy5 siRNA-OINCs, conjugate (INPs+cy5 siRNA) and complex (hiperfect+cy5 siRNA) at a dose of 25 nM siRNA. At various time points (2 h, 5 h, 24 h and 48 h) these cells were fixed with 4% paraformaldehyde, stained nuclei with DAPI and were then visualized by a fluorescence microscope (Carl Zeiss Axioplan, Germany). Scale bar is 10 p.m.

FIG. 13 shows mechanism of cellular uptake of OINCs. Ovarian cancer cells (OV90, 5×10⁴ cells/well in a 24 well plate) were cultured overnight in coverslips and were then treated with either in the presence or in the absence of chemical inhibitors at a concentration of 10 ug/ml (chlorpromazine), 10 uM (chloroquine), 5 ug/ml (filipin), 10 uM (rottlerin) and 5 uM (brefeldin) for 2 hr at 37° C. with 5% CO₂. Post 2 hr, cy5 CTL siRNA-ONCs and cy5 CTL siRNA-OINCs at a dose of 25 nM CTL siRNA were incubated for 4 hr at the same condition. These cells were fixed with 4% paraformaldehyde, stained nuclei with DAPI and were then visualized by fluorescence microscopy (Carl Zeiss Axioplan, Germany). Scale bar is 10 μm.

FIG. 14 shows quantitative cellular uptake of OINCs by CP20 cells. At the same conditions as used in the experiment in FIG. 12, cells were grown in 24 well plate and were incubated with cy5 siRNA, cy5 siRNA-ONCs, cy5 siRNA-OINCs, complex (hiperfect+cy5 siRNA) and conjugate (INPs+cy5 siRNA) at a final concentration of 25 nM siRNA. Cells were lysed after these periods, collected the supernatants after a brief centrifugation and were quantified the fluorescence intensity using a CLARIOstar plate reader (BMG Labtech, Ortenberg, Germany).

FIG. 15 shows quantification of OINCs uptake in the presence of small inhibitors. At the same conditions, as used in the experiment in FIG. 13, cells were grown in 24 well plate without coverslips and were then followed the same procedure without fixation. Cells were then lysed and collected the supernatants after a brief centrifugation and were measured the fluorescence intensity at λex./λem.=650/670 nm using a CLARIOstar plate reader. Data are represented as % uptake, mean±SD, n=3 which were calculated from the following formulae: % uptake=(measured fl. int. of sample with inhibitor/measured fl. int. of sample without inhibitor)×100.

FIG. 16 examines CBS silencing activity in ovarian cancer (OVCAR4) cells by various nanocomposite formulations. OVCAR4 cells (2×10⁵) were cultured overnight in the presence of 10% FBS media and were then treated with control siRNA-ONCs, OINCs, INPs, CBS siRNA-OINCs, CBS siRNA-ONCs, complex (Hiperfect+CBS siRNA) and conjugate (CBS siRNA-INP) at a final concentration of 25 nM siRNA. At 48 h post incubation, the extent of CBS silencing was determined at the protein level by western blot where GAPDH was used as a loading control.

FIG. 17 shows the stability of silencing activity of various formulations. OVCAR4 cells (2×10⁵) were cultured overnight in the presence of 10% FBS media and were then treated with OINCs, INPs, CBS siRNA-OINCs, CBS siRNA-ONCs, complex, control siRNA complex and empty-ONCs at a final concentration of 25 nM siRNA. At 6 h, 1 D, 2 D, 4 D, 7 D and 14 D post incubation, the extent of CBS silencing was determined at the protein level by western blot. GAPDH was used as a loading control.

FIG. 18 shows the effects of various OINC formulations on cell viability. OVCAR4 cells (lower panel) and OV90 cells (upper panel) were grown overnight in 96 well plates at a density of 3000 cells/well and were then treated with control siRNA-ONCs, OINCs, INPs, CBS siRNA-OINCs, CBS siRNA-ONCs, CBS siRNA-INPs conjugate at a dose of 25 nM and complex (25 or 133 nM) or were untreated. After 48 h cell viability was measured by the MTT assay (n=6).

FIG. 19 shows the effects of various OINC formulations on clonal growth. OVCAR4 cells (lower panel) and OV90 cells (upper panel) (200 cells/35 mm dish) were co-transfected with either control siRNA-ONCs, OINCs, INPs, CBS siRNA-OINCs, CBS siRNA-ONCs, complex and conjugate at the same dose or complex at a dose of 133 nM or remains untreated. After 12 (OVCAR4) or 8 (OV90) days, colonies were stained with crystal violet, imaged and counted by using colony counter machine (n=3).

FIG. 20A shows the physicochemical characterizations of different sizes, shape and other type of inorganic nanoparticles-MICU1 siRNA loaded. ONCs. The sizes was measured by dynamic light scatter microscopy (DLS) (n=3).

FIG. 20B shows the physicochemical zeta characterizations of different sizes, shape and other type of inorganic nanoparticles-MICU1 siRNA loaded ONCs. The zeta potentials were measured by dynamic light scatter microscopy (DLS) (n=3).

FIG. 20C shows encapsulation efficiency as a percent entrapment of MICU1 siRNA in ONCs of different sizes, shapes and other type of inorganic nanoparticles. Entrapment was measured using the ribogreen assay (n=3).

FIG. 21A exhibits the effects of various sizes, shape and other type inorganic nanoparticles incorporation on MICU1 silencing activity. OV90 cells (1.5×10⁵) were cultured overnight in the presence of 10% FBS media and were then treated with either control siRNA-OINCs, siMICU1-OINCs or siMICU1-INPs conjugate of various sizes of INPs (5, 20 and 50 nm) at a final concentration of 50 nM siRNA. At 72 h post incubation, the extent of MICU1 silencing was determined at the protein level by western blot where GAPDH was used as a loading control and a quantitative measurement MICU1 protein expression is also shown, analyzed by Image J, percent silencing activity of MICU1 protein is compared to that of control siRNA, normalized by GAPDH (n=3).

FIG. 21B exhibits the effects of rod shape inorganic nanoparticles incorporation on MICU1 silencing activity. OV90 cells (1.5×10⁵) were cultured overnight in the presence of 10% FBS media and were then treated with either control siRNA-OINCs, siMICU1-OINCs and siMICU1-INPs conjugate of rod shape INPs (25 nm) or control siRNA-OINCs, siMICU1-OINCs and siMICU1-INPs conjugate of 20 nm oval shape INPs at a final concentration of 50 nM siRNA. At 72 h post incubation, the extent of MICU1 silencing was determined at the protein level by western blot where GAPDH was used as a loading control and a quantitative measurement MICU1 protein expression is also shown, analyzed by Image J, percent silencing activity of MICU1 protein is compared to that of control siRNA, normalized by GAPDH (n=3).

FIG. 21C exhibits the effects of 20 nm magnetic inorganic nanoparticles incorporation on MICU1 silencing activity. OV90 cells (1.5×10⁵) were cultured overnight in the presence of 10% FBS media and were then treated with either control siRNA-OINCs, siMICU1-OINCs and siMICU1-INPs conjugate of Fe₃O₄-INPs (20 nm) or control siRNA-OINCs, siMICU1-OINCs and siMICU1-INPs conjugate of 20 nm INPs at a final concentration of 50 nM siRNA. At 72 h post incubation, the extent of MICU1 silencing was determined at the protein level by western blot where GAPDH was used as a loading control and a quantitative measurement MICU1 protein expression is also shown, analyzed by Image J, percent silencing activity of MICU1 protein is compared to that of control siRNA, normalized by GAPDH (n=3).

FIG. 22 characterizes MICU1 siRNA ONCs and OINCs according to size (left), zeta potential (center), and percent entrapment (right) of the siRNA. The size and zeta potential were measured by dynamic light scatter microscopy (DLS) and entrapment was measured using the ribogreen assay (n=3).

FIG. 23 Morphology of MICU1 siRNA-ONCs and MICU1 siRNA-OINCs was observed by using transmission electron microscopy (TEM).

FIG. 24 shows serum stability results of MICU1 siRNA-OINCs. MICU1 siRNA, MICU1 siRNA-ONCs, MICU1 siRNA-OINCs, complex (Hiperfect+MICU1 siRNA) and conjugate (INPs+MICU1 siRNA) (1.5 ug each) incubated with 100% FBS (1:1 v/v) at 37° C. for 4 day. 1.5% agarose gel electrophoresis was performed in the presence of TBE buffer.

FIG. 25 shows the level of silencing activity of various MICU1 siRNA formulations. OV90 cells (1.5×10⁵) were cultured overnight in the presence of 10% FBS media and were then treated with control siRNA-OINCs, INPs, CLT siRNA complex, MICU1 siRNA-OINCs, MICU1 siRNA-ONCs, conjugate at a final concentration of 50 nM siRNA and complex at doses of 50 and 133 nM MICU1 siRNA. At 72 h post incubation, the extent of MICU1 silencing was determined at the mRNA level using RT-PCR. Relative mRNA level was normalized to the GAPDH reference gene.

FIG. 26 shows the effects of various OINC formulations on cell viability. OV90 cells were grown in 96 well plate at a density of 2500-3000 cells/well overnight and were then treated either control siRNA-OINCs, INPs, CLT siRNA complex, MICU1 siRNA-OINCs, MICU1 siRNA-ONCs, complex and conjugate at the same dose or complex at a dose of 133 nM or remains untreated. After 48 h and 72 h, cells viability was measured by the MTT assay (n=5).

FIG. 27 shows the effects of various OINC formulations on clonal growth. OV90 cells (200 cells/35 mm dish) were co-transfected with either control siRNA-OINCs, INPs, CLT siRNA complex, MICU1 siRNA-OINCs, MICU1 siRNA-ONCs, complex and conjugate at the same dose or complex at a dose of 133 nM or remained untreated. After 12 days, colonies were stained with crystal violet, imaged and counted by using colony counter machine (n=3).

FIG. 28 shows results of ex vivo tumor homing of fluorescence labeled CTL siRNA-OINCs. Ex vivo images of ovarian tumor and whole body organs. Tumor bearing athymic nude mice (n=4) were i.v injected with either 5 ug of Cy5 CLT siRNA-ONCs (right panel), Cy5 CLT siRNA-OINCs (left panel) or remained untreated and at 24 h, tissues (tumor, liver, spleen, kidneys, lungs and heart) were imaged by using Carestream Xtreme In Vivo Imaging System.

FIG. 29 shows quantitative results of the experiment of FIG. 28 as measured by fluorescence intensity of the accumulated nanoparticles by Image J (a) and % injected dose (% ID) accumulated in tumor (b).

FIG. 30 shows results of in vivo tumor therapy of MICU1 siRNA-OINCs. Tumor harboring athymic nude mice (n=5) were i.v injected with either 5 μg of MICU1 siRNA-ONCs, MICU1 siRNA-OINCs or CLT siRNA-OINCs every 4 days for a total period of 12 days. Tumor size was measured following treatment. Individual tumors were measured using a vernier calliper every 2-day and tumor volume was calculated by: tumor volume (mm³)=length×(width)/2.

FIG. 31 shows representative images of the tumor therapy of the experiment of FIG. 30.

FIG. 32 shows tumor mass (a) and body weight (b) after 12 days in the animals of experiment of FIG. 30.

FIG. 33 shows measurements of MICU1 protein expression in tumor tissues using western blot analysis after 12 days of treatment in the animals of the experiment of FIG. 30. GAPDH was used as an internal control.

FIG. 34 shows measurements of relative MICU1 mRNA levels after 12 days in the animals of the experiment of FIG. 30.

FIG. 35 shows representative CD31 stained sections of tumors from siCTL-OINCs, siMICU1-ONCs and siMICU1-OINCs groups (a) and quantification of CD31 stained vessels, analyzed by Image J was expressed as a fold decrease vascular density, compared to siCTL-OINCs group (b), n=6 of each mouse tissue. Scale bar is 50 μm.

FIG. 36 shows representative Ki67 stained sections of tumors from siCTL-OINCs, siMICU1-ONCs and siMICU1-OINCs groups (a) and quantification of Ki67 stained proliferating cells, analyzed by Image J was expressed as percentage (%) decrease, compared to siCTL-OINCs group (b), n=6 of each mouse tissue. Scale bar is 50 μm.

FIG. 37 shows representative TUNEL (+) Ve cells (red) stained sections of tumors from siCTL-OINCs, siMICU1-ONCs and siMICU1-OINCs groups (a) and quantification of TUNEL (+) Ve cells, analyzed by Image J was expressed as a fold increase apoptotic cell signal, compared to siCTL-OINCs group (b), n=6 of each mouse tissue. Scale bar is 50 μm.

FIG. 38 shows representative H & E stained sections of tissues (tumor, liver, spleen, lungs, kidneys and heart) from siCTL-OINCs, siMICU1-ONCs and siMICU1-OINCs groups where black arrow heads indicates hepatic toxicity (granuloma) in liver and inflammation in lung of siMICU1-ONCs group. Scale bar is 50 μm.

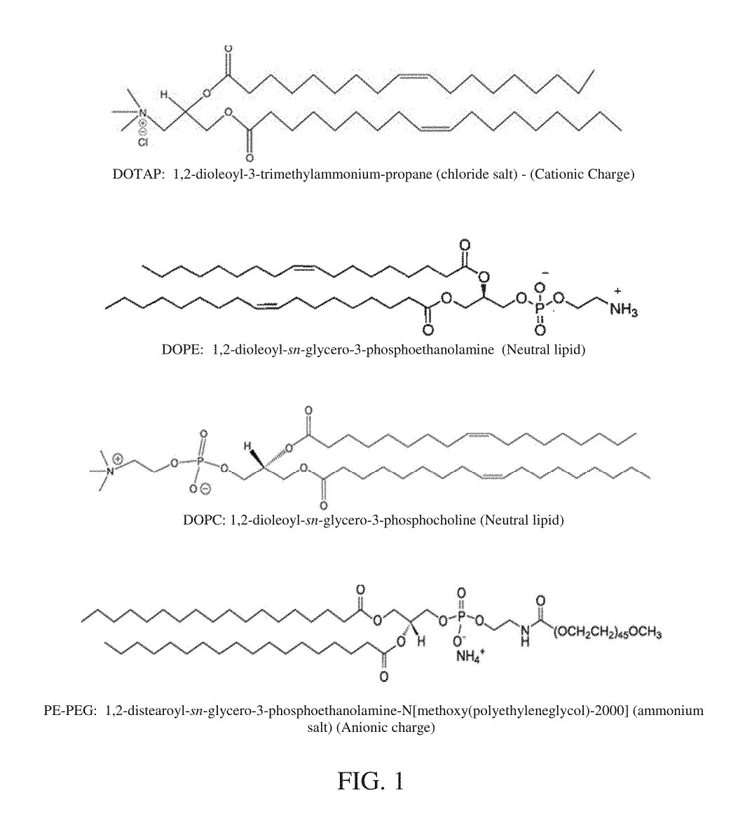

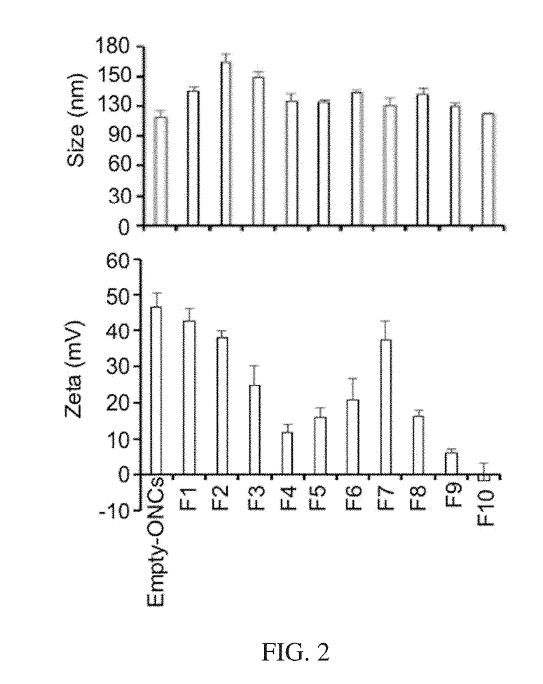

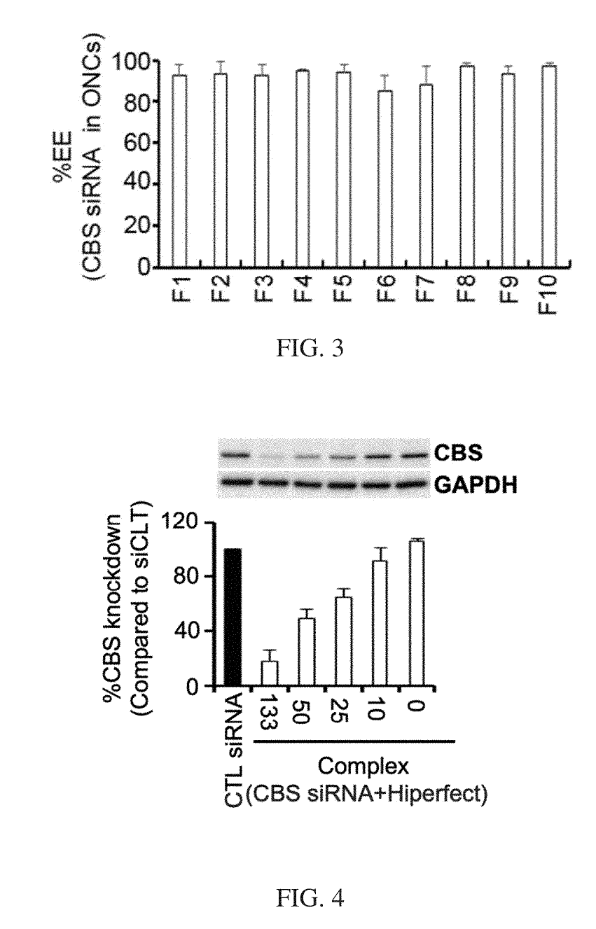

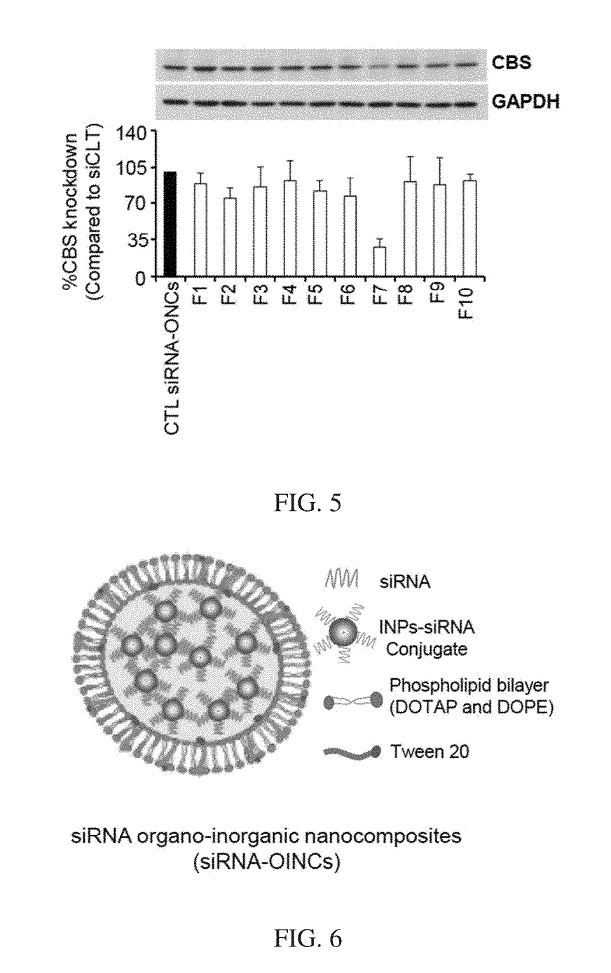

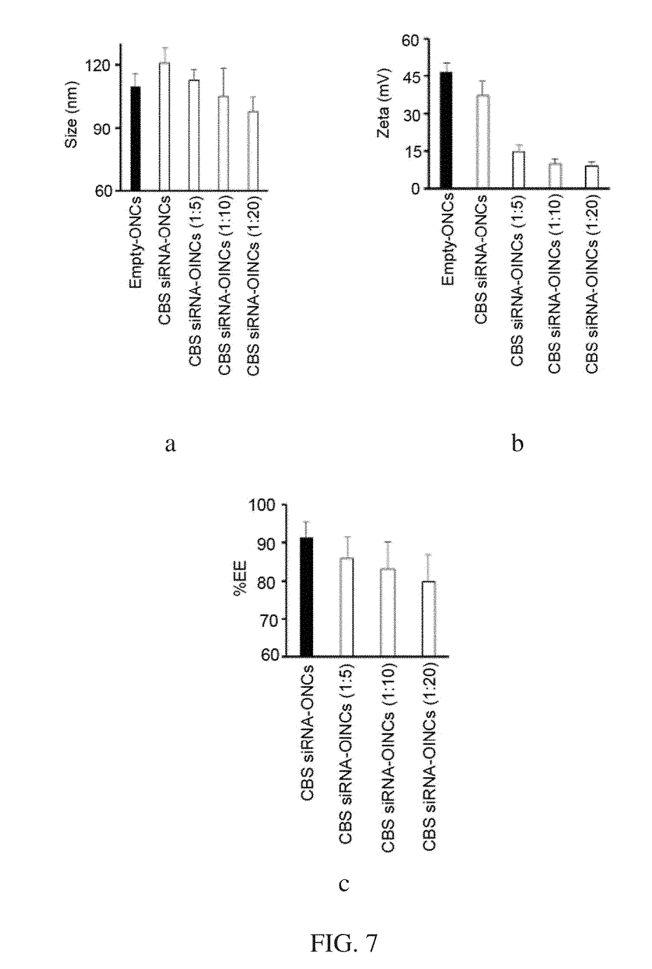

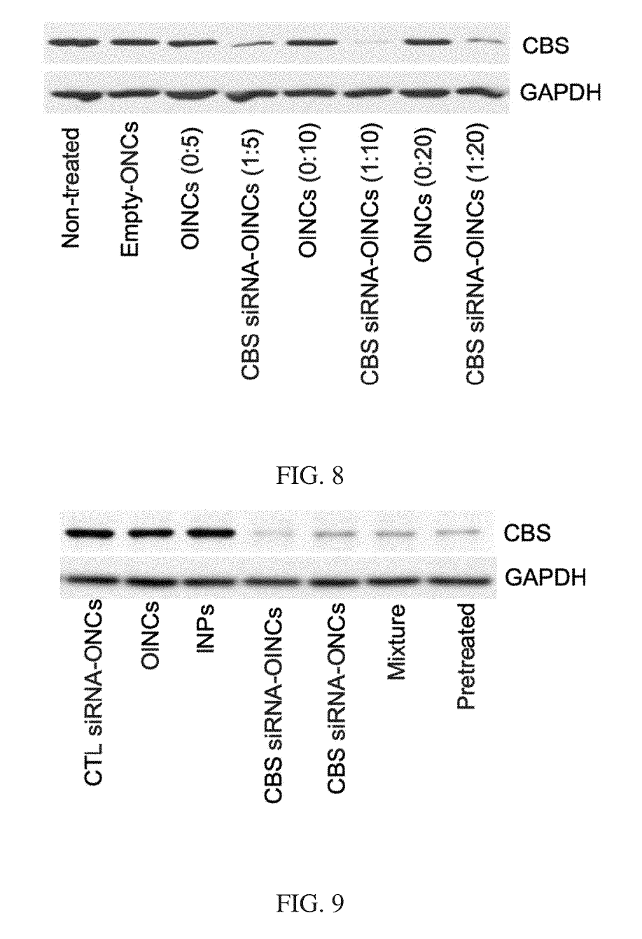

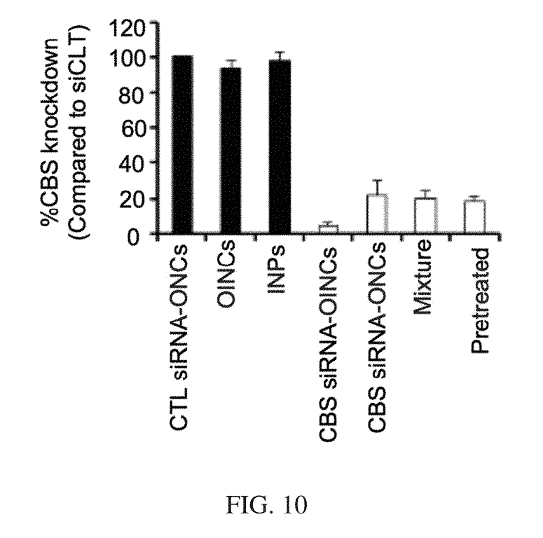

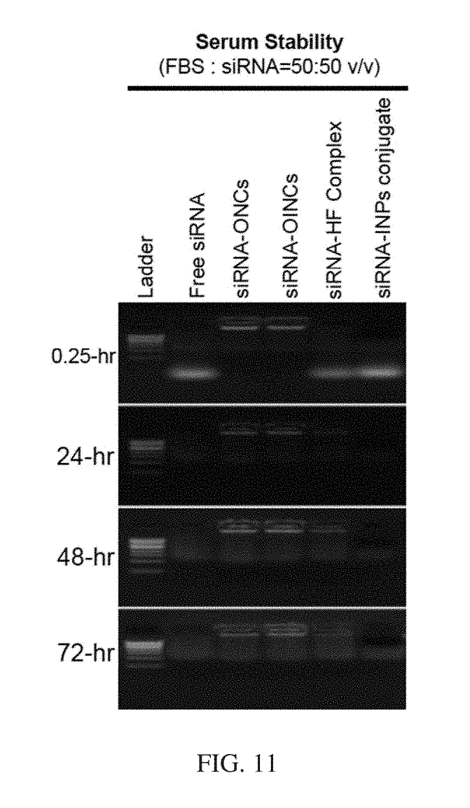

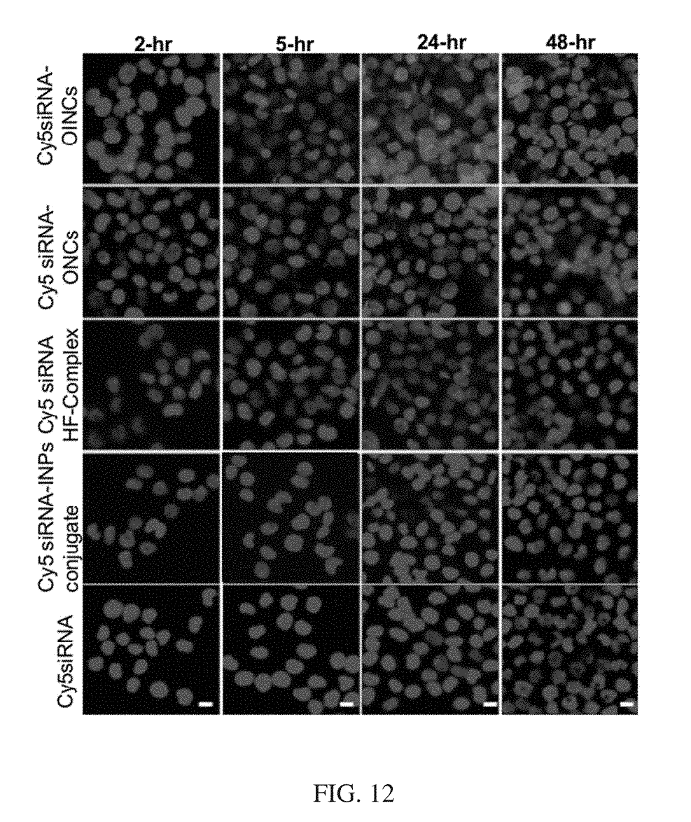

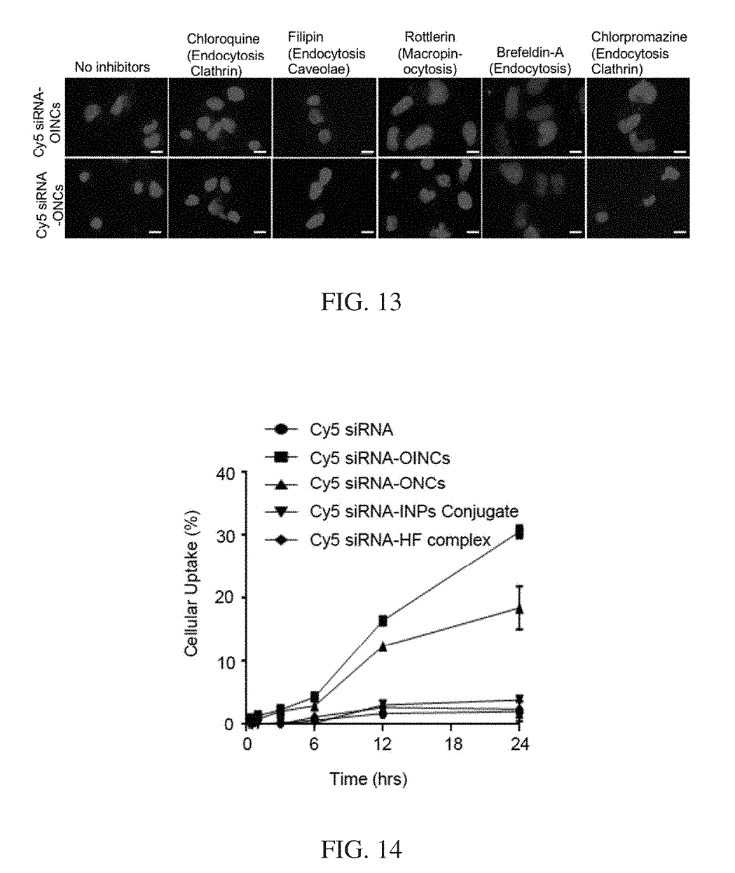

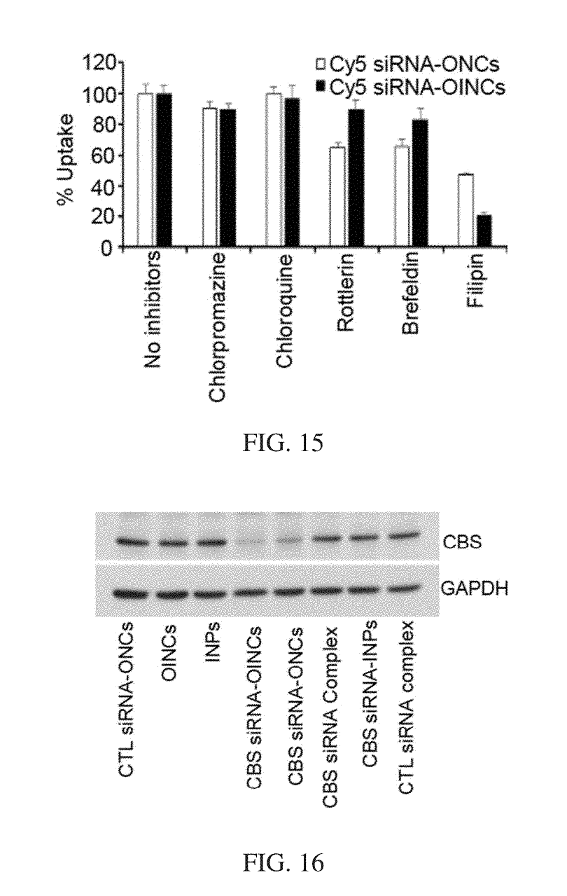

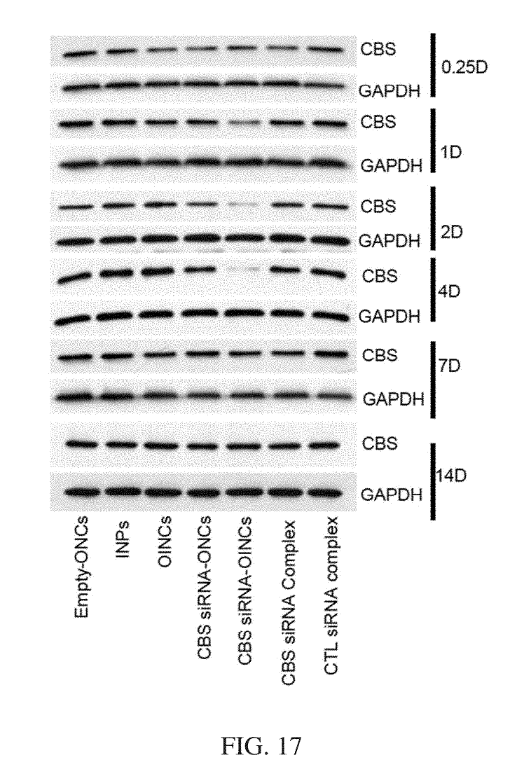

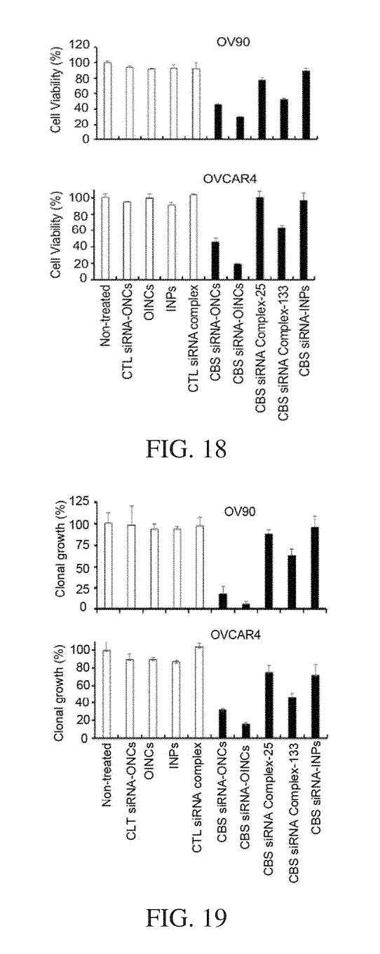

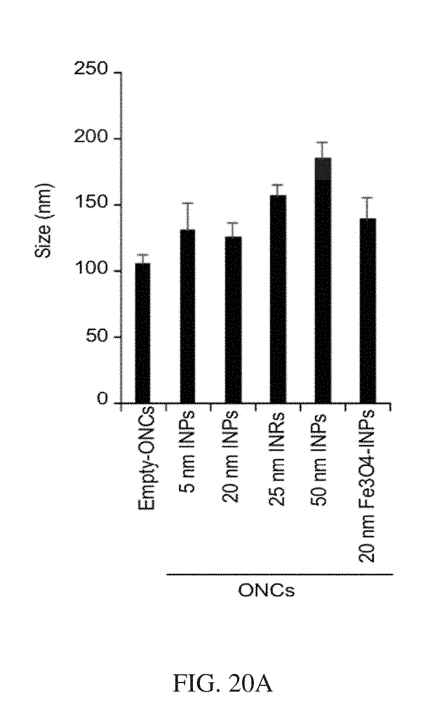

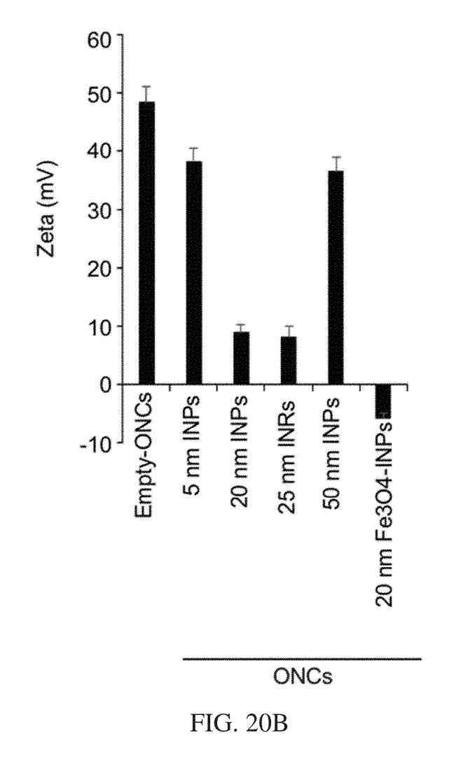

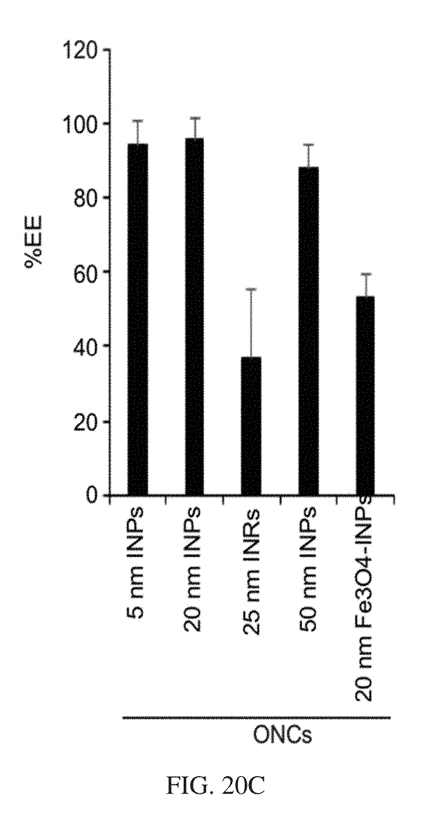

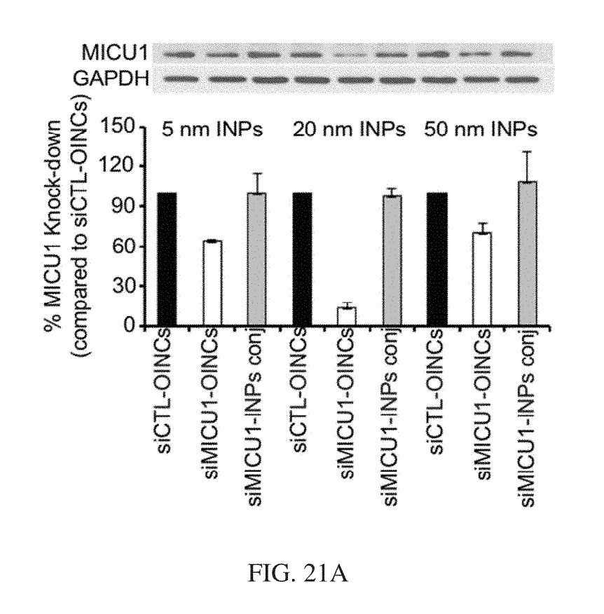

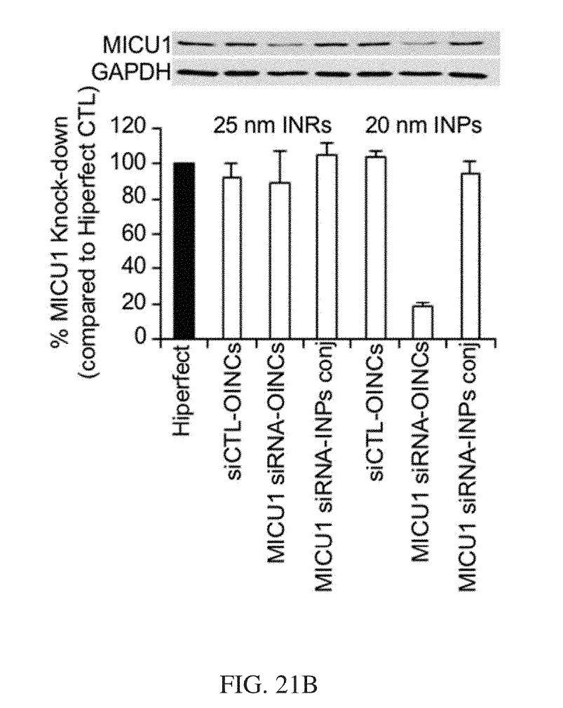

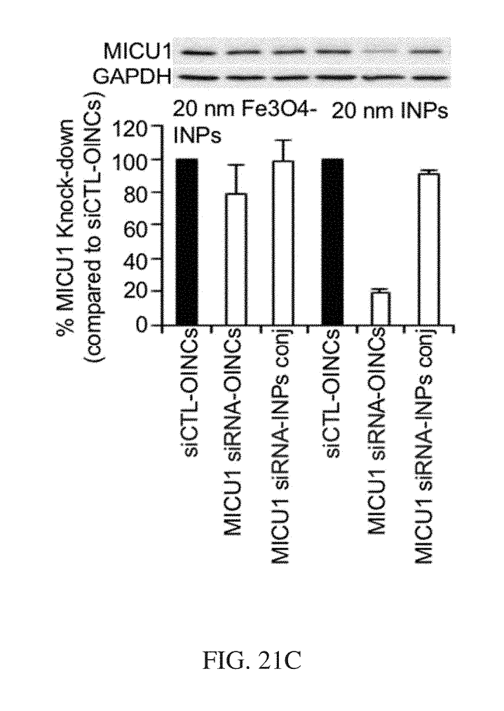

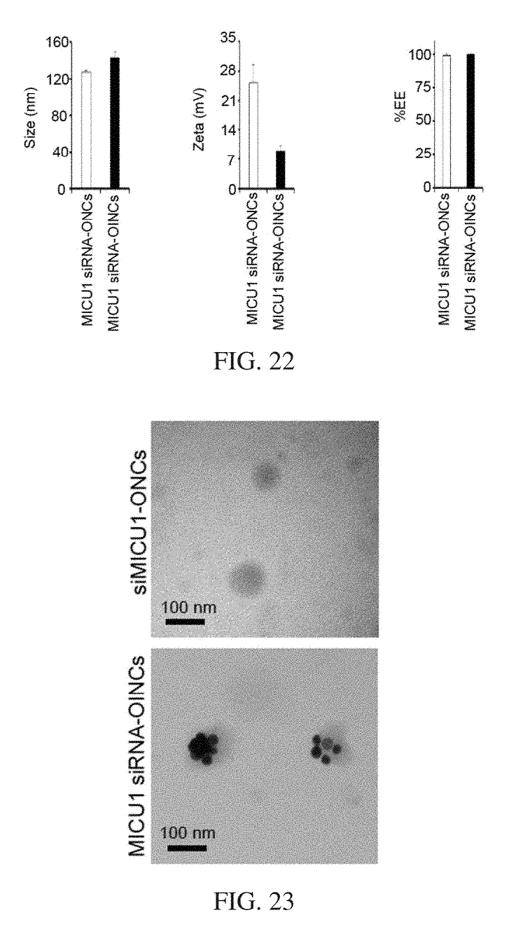

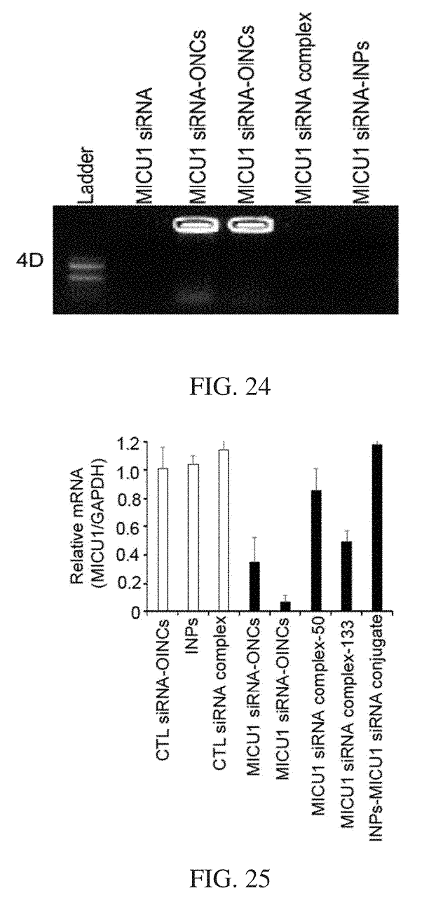

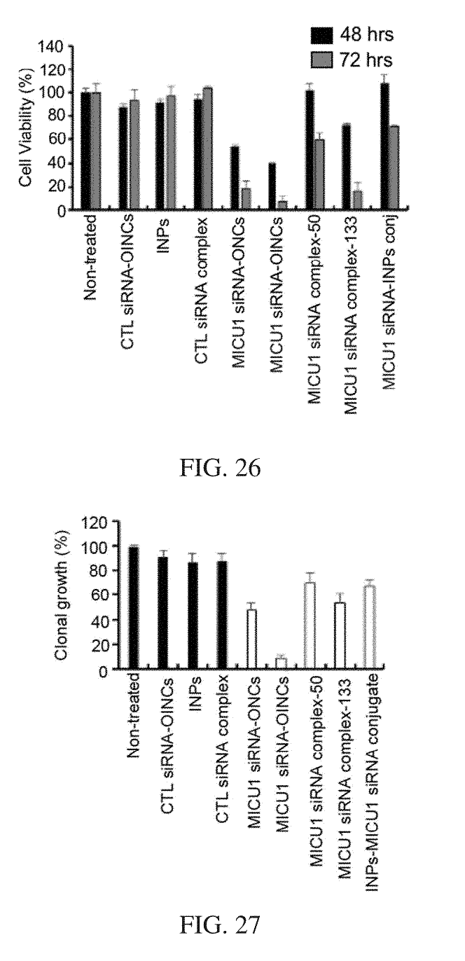

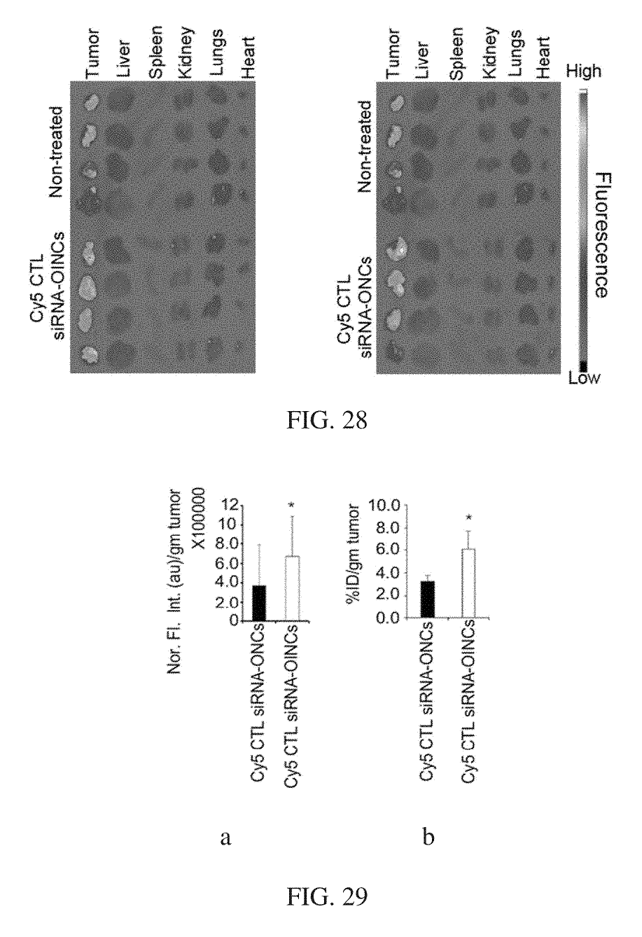

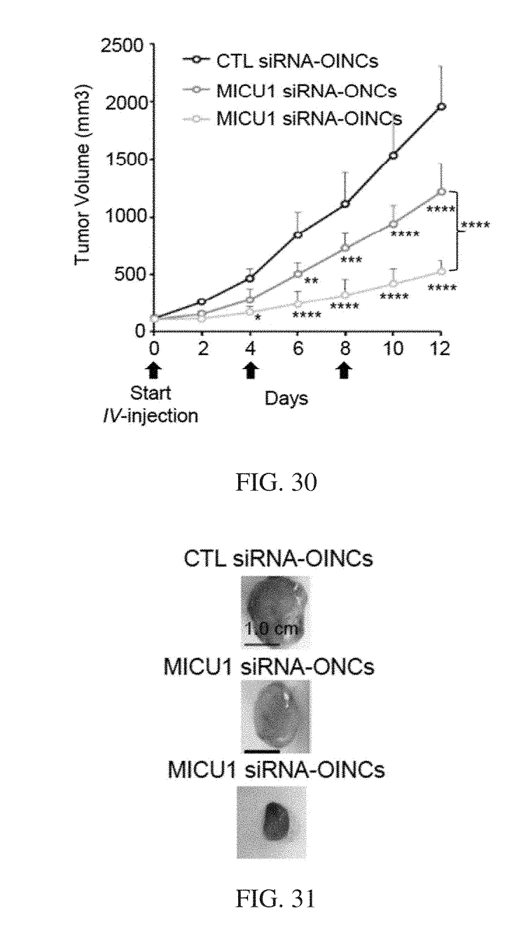

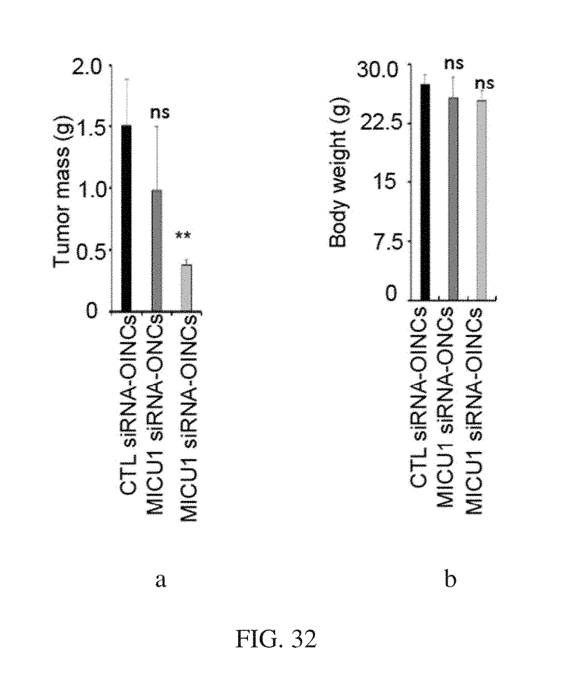

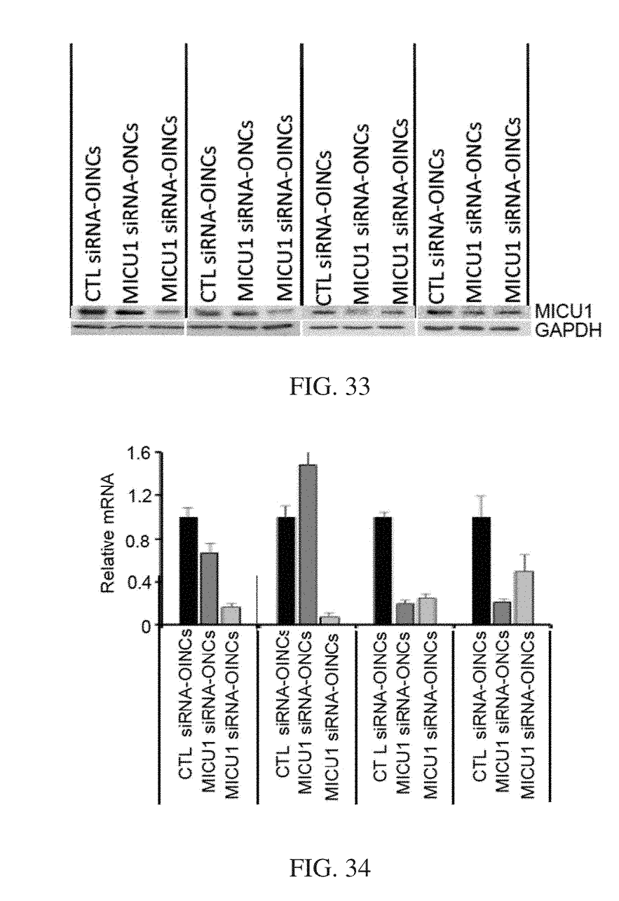

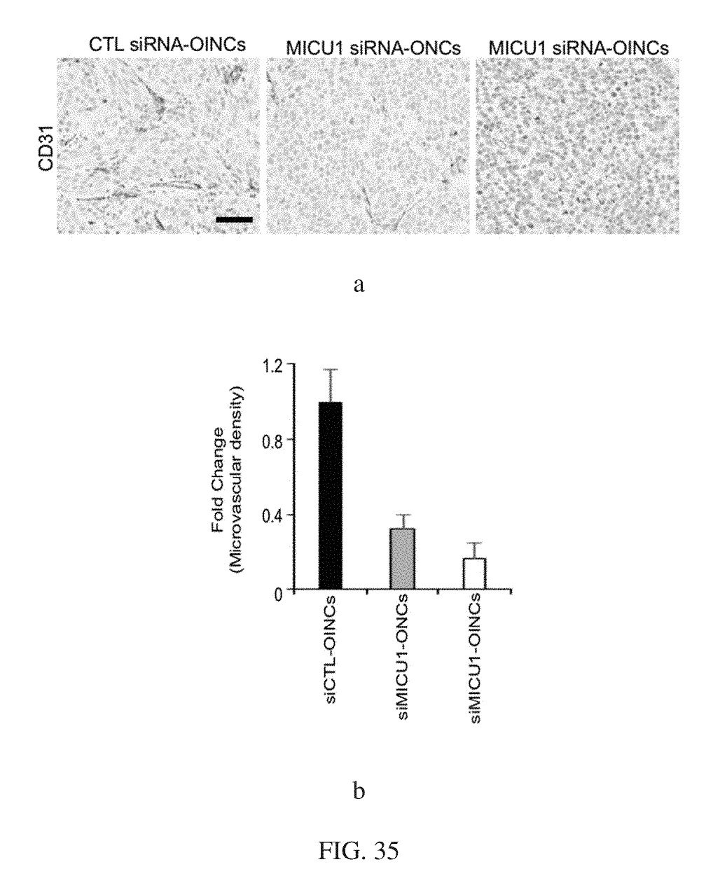

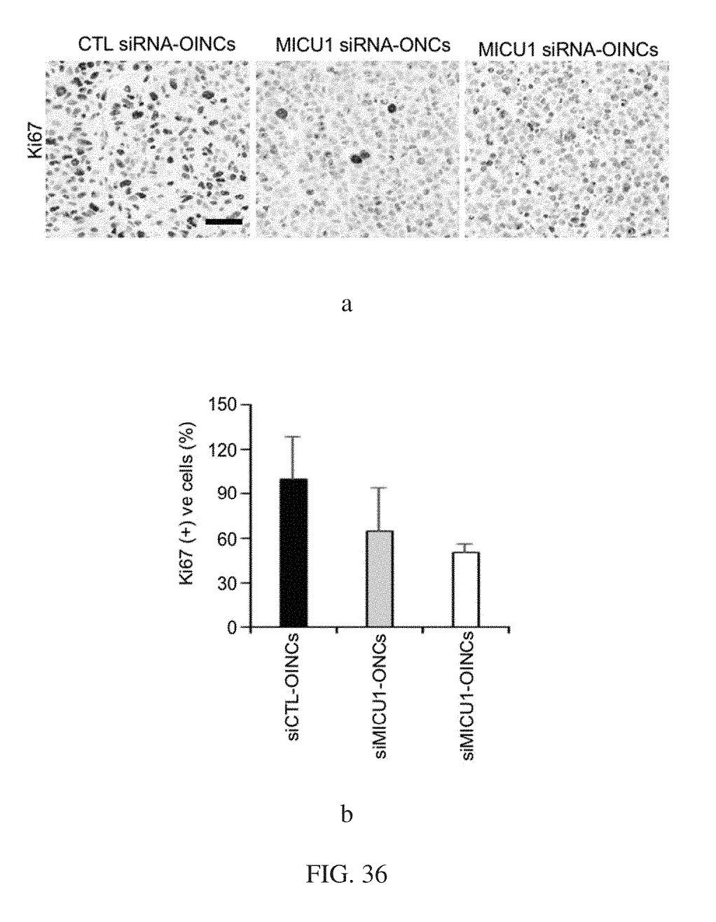

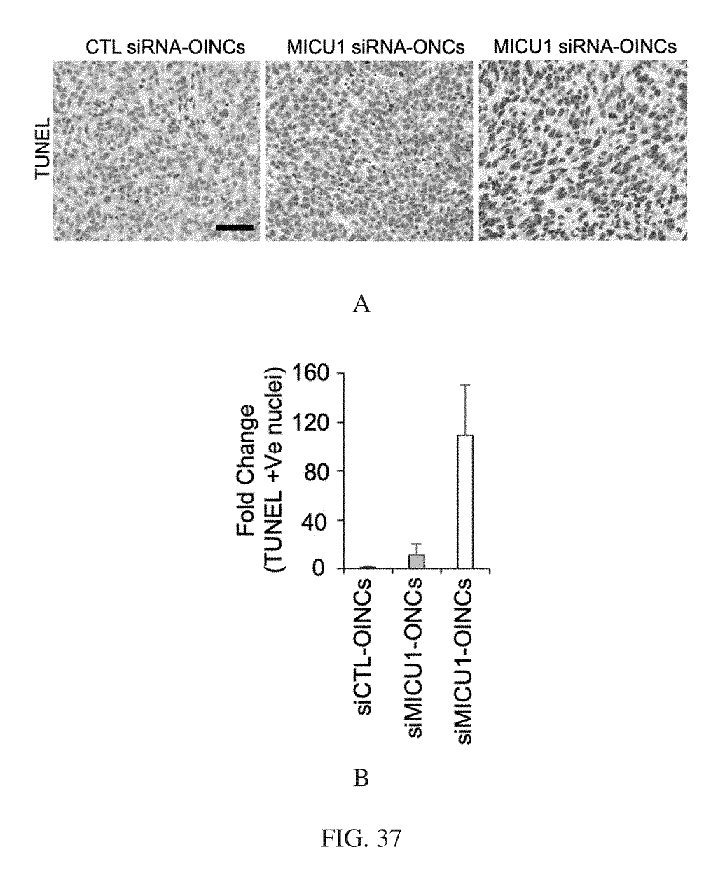

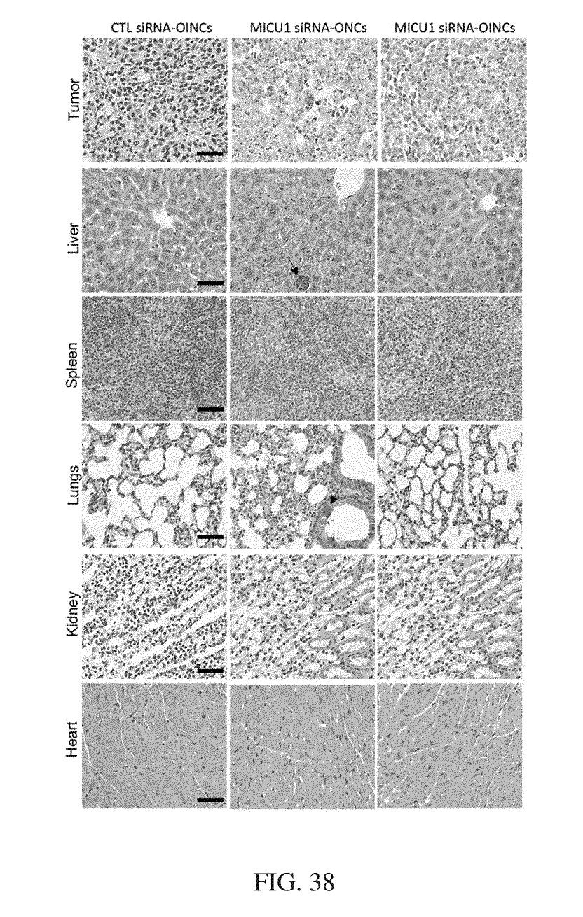

### DETAILED DESCRIPTION

The present disclosure is directed to a composition and method for the delivery of a cargo molecule (e.g., a therapeutic molecule, or agent useful in diagnosis or imaging), including but not limited to siRNA, miRNA, peptides, proteins, and small molecules such as fluorescently labelled dyes. In certain embodiments, the composition is a biocompatible organic nanocomposite (ONC) or an organo-inorganic nanocomposite (OINC). The ONC can comprise a lipid component (e.g., at least one or more lipid bilayers forming an outer lipid shell) which includes a cationic lipid, a non-cationic fusogenic co-lipid, and a pore-forming agent such as a surfactant, and an encapsulated core comprising a cargo molecule. The term non-cationic refers to either neutrally-charged or anionically-charged lipids. The encapsulated core containing the cargo molecule may be aqueous. The OINC can comprise a lipid component (e.g., at least one or more lipid bilayers forming an outer lipid shell) which includes a cationic lipid, a non-cationic fusogenic co-lipid, and a pore-forming molecule such as a surfactant, and a cargo-inorganic component including an inorganic nanoparticle, such as gold nanoparticle, which is conjugated (bound) to the organic cargo molecule directly via a covalent bond (such as a thiol or amine), or indirectly via electrostatic forces. The encapsulated core containing the cargo-inorganic component may be aqueous. The ONC or OINC may be used, for example, for treating a disease, such as cancer or any other disease or condition which responds to a therapy, or benefits from diagnostic or imaging techniques.

Before further describing various embodiments of the compositions and methods of the present disclosure in more detail by way of exemplary description, examples, and results, it is to be understood that the embodiments of the present disclosure are not limited in application to the details of methods and compositions as set forth in the following description. The embodiments of the compositions and methods of the present disclosure are capable of being practiced or carried out in various ways not explicitly described herein. As such, the language used herein is intended to be given the broadest possible scope and meaning; and the embodiments are meant to be exemplary, not exhaustive. Also, it is to be understood that the phraseology and terminology employed herein is for the purpose of description and should not be regarded as limiting unless otherwise indicated as so. Moreover, in the following detailed description, numerous specific details are set forth in order to provide a more thorough understanding of the disclosure. However, it will be apparent to a person having ordinary skill in the art that the embodiments of the present disclosure may be practiced without these specific details. In other instances, features which are well known to persons of ordinary skill in the art have not been described in detail to avoid unnecessary complication of the description. All of the compositions and methods of production and application and use thereof disclosed herein can be made and executed without undue experimentation in light of the present disclosure. While the compositions and methods of the present disclosure have been described in terms of particular embodiments, it will be apparent to those of skill in the art that variations may be applied to the compositions and/or methods and in the steps or in the sequence of steps of the method described herein without departing from the concept, spirit, and scope of the inventive concepts as described herein. All such similar substitutes and modifications apparent to those having ordinary skill in the art are deemed to be within the spirit and scope of the inventive concepts as disclosed herein.

All patents, published patent applications, and non-patent publications including articles referenced or mentioned in any portion of the present specification are indicative of the level of skill of those skilled in the art to which the present disclosure pertains, and are hereby expressly incorporated by reference in their entirety to the same extent as if the contents of each individual patent or publication was specifically and individually incorporated herein.

Unless otherwise defined herein, scientific and technical terms used in connection with the present disclosure shall have the meanings that are commonly understood by those having ordinary skill in the art. Further, unless otherwise required by context, singular terms shall include pluralities and plural terms shall include the singular.

As utilized in accordance with the methods and compositions of the present disclosure, the following terms, unless otherwise indicated, shall be understood to have the following meanings:

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or when the alternatives are mutually exclusive, although the disclosure supports a definition that refers to only alternatives and “and/or.” The use of the term “at least one” will be understood to include one as well as any quantity more than one, including but not limited to, 2, 3, 4, 5, 6, 7, 8, 9, 10, 15, 20, 30, 40, 50, 100, or any integer inclusive therein. The term “at least one” may extend up to 100 or 1000 or more, depending on the term to which it is attached; in addition, the quantities of 100/1000 are not to be considered limiting, as higher limits may also produce satisfactory results. In addition, the use of the term “at least one of X, Y and Z” will be understood to include X alone, Y alone, and Z alone, as well as any combination of X, Y and Z.

As used in this specification and claims, the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AAB, BBC, AAABCCCC, CBBAAA, CABABB, and so forth. The skilled artisan will understand that typically there is no limit on the number of items or terms in any combination, unless otherwise apparent from the context.

Throughout this application, the term “about” is used to indicate that a value includes the inherent variation of error for the composition, the method used to administer the composition, or the variation that exists among the objects, or study subjects. As used herein the qualifiers “about” or “approximately” are intended to include not only the exact value, amount, degree, orientation, or other qualified characteristic or value, but are intended to include some slight variations due to measuring error, manufacturing tolerances, stress exerted on various parts or components, observer error, wear and tear, and combinations thereof, for example. The term “about” or “approximately”, where used herein when referring to a measurable value such as an amount, a temporal duration, and the like, is meant to encompass, for example, variations of ±20% or ±10%, or ±5%, or ±1%, or ±0.1% from the specified value, as such variations are appropriate to perform the disclosed methods and as understood by persons having ordinary skill in the art. As used herein, the term “substantially” means that the subsequently described event or circumstance completely occurs or that the subsequently described event or circumstance occurs to a great extent or degree. For example, the term “substantially” means that the subsequently described event or circumstance occurs at least 90% of the time, or at least 95% of the time, or at least 98% of the time.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth. Reference to a series of ranges includes ranges which combine the values of the boundaries of different ranges within the series. Thus, to illustrate reference to a series of ranges, for example, a range of 1-1,000 includes, for example, 1-10, 10-20, 20-30, 30-40, 40-50, 50-60, 60-75, 75-100, 100-150, 150-200, 200-250, 250-300, 300-400, 400-500, 500-750, 750-1,000, and includes ranges of 1-20, 10-50, 50-100, 100-500, and 500-1,000. Any two values within the above ranges, e.g., 88 and 444 therefore can be used to set the lower and upper boundaries of a range (e.g., 88-444) in accordance with the embodiments of the present disclosure.

The term “pharmaceutically acceptable” refers to compounds and compositions which are suitable for administration to humans and/or animals without undue adverse side effects such as toxicity, irritation and/or allergic response commensurate with a reasonable benefit/risk ratio. The term “biocompatible” has the same meaning as “pharmaceutically acceptable” and may be used interchangeably therewith.

By “biologically active” is meant the ability to modify the physiological system of an organism without reference to how the active agent has its physiological effects.

As used herein, “pure, “substantially pure,” or “isolated” means an object species is the predominant species present (i.e., on a molar basis it is more abundant than any other object species in the composition thereof), and particularly a substantially purified fraction is a composition wherein the object species comprises at least about 50 percent (on a molar basis) of all macromolecular species present. Generally, a substantially pure composition will comprise more than about 80% of all macromolecular species present in the composition, more particularly more than about 85%, more than about 90%, more than about 95%, or more than about 99%. The term “pure” or “substantially pure” also refers to preparations where the object species (e.g., the peptide compound) is at least 60% (w/w) pure, or at least 70% (w/w) pure, or at least 75% (w/w) pure, or at least 80% (w/w) pure, or at least 85% (w/w) pure, or at least 90% (w/w) pure, or at least 92% (w/w) pure, or at least 95% (w/w) pure, or at least 96% (w/w) pure, or at least 97% (w/w) pure, or at least 98% (w/w) pure, or at least 99% (w/w) pure, or 100% (w/w) pure. Where used herein the term “high specificity” refers to a specificity of at least 90%, or at least 91%, or at least 92%, or at least 93%, or at least 94%, or at least 95%, or at least 96%, or at least 97%, or at least 98%, or at least 99%. Where used herein the term “high sensitivity” refers to a sensitivity of at least 90%, or at least 91%, or at least 92%, or at least 93%, or at least 94%, or at least 95%, or at least 96%, or at least 97%, or at least 98%, or at least 99%.

The terms “subject” and “patient” are used interchangeably herein and will be understood to refer to a warm blooded animal, particularly a mammal or bird. Non-limiting examples of animals within the scope and meaning of this term include dogs, cats, rats, mice, guinea pigs, horses, goats, cattle, sheep, zoo animals, Old and New World monkeys, non-human primates, and humans.

“Treatment” refers to therapeutic treatments. “Prevention” refers to prophylactic treatment measures to stop a condition from occurring. The term “treating” refers to administering the composition to a patient for therapeutic purposes, and may result in an amelioration of the condition or disease.

The terms “therapeutic composition” and “pharmaceutical composition” refer to an active agent-containing composition that may be administered to a subject by any method known in the art or otherwise contemplated herein, wherein administration of the composition brings about a therapeutic effect as described elsewhere herein. In addition, the compositions of the present disclosure may be designed to provide delayed, controlled, extended, and/or sustained release using formulation techniques which are well known in the art.

The term “effective amount” refers to an amount of an active agent which is sufficient to exhibit a detectable biochemical and/or therapeutic effect, for example without excessive adverse side effects (such as toxicity, irritation and allergic response) commensurate with a reasonable benefit/risk ratio when used in the manner of the inventive concepts. The effective amount for a patient will depend upon the type of patient, the patient's size and health, the nature and severity of the condition to be treated, the method of administration, the duration of treatment, the nature of concurrent therapy (if any), the specific formulations employed, and the like. Thus, it is not possible to specify an exact effective amount in advance. However, the effective amount for a given situation can be determined by one of ordinary skill in the art using routine experimentation based on the information provided herein.

The term “ameliorate” means a detectable or measurable improvement in a subject's condition or or symptom thereof. A detectable or measurable improvement includes a subjective or objective decrease, reduction, inhibition, suppression, limit or control in the occurrence, frequency, severity, progression, or duration of the condition, or an improvement in a symptom or an underlying cause or a consequence of the condition, or a reversal of the condition. A successful treatment outcome can lead to a “therapeutic effect,” or “benefit” of ameliorating, decreasing, reducing, inhibiting, suppressing, limiting, controlling or preventing the occurrence, frequency, severity, progression, or duration of a condition, or consequences of the condition in a subject. A decrease or reduction in worsening, such as stabilizing the condition, is also a successful treatment outcome. A therapeutic benefit therefore need not be complete ablation or reversal of the condition, or any one, most or all adverse symptoms, complications, consequences or underlying causes associated with the condition. Thus, a satisfactory endpoint may be achieved when there is an incremental improvement such as a partial decrease, reduction, inhibition, suppression, limit, control or prevention in the occurrence, frequency, severity, progression, or duration, or inhibition or reversal of the condition (e.g., stabilizing), over a short or long duration of time (e.g., seconds, minutes, hours).

Returning now to the various embodiments of the present disclosure, in at least certain embodiments, the ONCs or OINC particles have a substantially net-neutral charge (i.e., a charge <10 mV). In certain embodiments of the OINC, at least 50% to 90% of the cargo-inorganic component is contained within the core (inner sphere) of the OINC particle, i.e., a portion of the cargo-inorganic component is exposed in the outer lipid layer. In certain embodiments of the OINC, the cargo-inorganic component is substantially encapsulated (i.e., at least 90% to 99%) within the core (inner sphere) of the OINC particle (e.g., at least 90%-95%). Therefore, in at least certain embodiments, at least a portion of the cargo-inorganic component is present in the lipid outer layer, wherein the resulting OINC particle possesses a substantially net neutral charge, which enhances delivery of the cargo to cells in vitro and in vivo as compared to, for example, certain positively-charged liposomal particles (ONCs). Examples of lipids that can be used in the formation of the lipid components of the various nanocomposites of the present disclosure include, but are not limited to, those described in U.S. Pat. No. 9,616,020, and U.S. Patent Publication Nos. 20180021453, 20170232115, and 20170105936. Examples of pore-forming agents that can be used in accordance with the present disclosure include, but are not limited to, pore-forming surfactants. Examples of pore-forming surfactants include but are not limited to Tween 20, Triton X-100, Brij 56, pluronic F127, polyethylene glycols, and polypropylene glycols.

In at least one embodiment disclosed herein, the inorganic nanoparticles of the OINC are gold nanoparticles (AuNP or GNP), such as described in U.S. Pat. Nos. 9,382,346, 9,605,304, and 9,719,089, including AuNPs having of different sizes (e.g., diameters in a range of 5 nm to 50 nm, such as 5 nm, 20 nm and 50 nm), shape (e.g., 25 nm gold nanorod) and other types of similarly sized inorganic nanoparticle (e.g., 20 nm magnetic nanoparticles), For example, where 20 nm AuNP incorporation shows the substantial potential to silence the target gene (e.g. MICU1). In a non-limiting embodiment, the AuNP is citrate-capped and net-negatively-charged particle. In a non-limiting embodiment, the OINCs have an average diameter in a range of 100 nm to 200 nm.

As noted above, the ONC or OINC may be used, for example, for treating a disease, such as cancer or any other disease or condition which responds to a therapy or benefits from diagnostic or imaging techniques. In at least certain embodiments, the ONC or OINC as disclosed herein has a prolonged serum stability and integrity. In certain embodiments, the present disclosure describes a method of delivering cargo molecules to cells of tumor sites by administering OINCs as described elsewhere herein to a subject in need of such therapy. The composition may be administered intravenously or by any other effective method. By this method, the OINC is useful to deliver an effective amount of a therapeutic molecule that can attenuate, slow, reduce or eliminate a condition or disease state in a subject or can treat a disease such as cancer, where the OINC can be delivered in a pharmaceutically acceptable vehicle. Examples of cancers that can be treated by the methods described herein include, but are not limited to, those described in U.S. Pat. No. 9,616,020 and U.S. Patent Publication Nos. 20170232115 and 20170105936. In certain embodiments, the cargo molecule is siRNA that inhibits translation of a gene (e.g., CBS or MICU1) that is overexpressed in the cancerous cell. Examples of methods of administrating the compositions disclosed herein, and dosages thereof, include, but are not limited to, those described in U.S. Pat. No. 9,616,020 and U.S. Patent Publication Nos. 20180021453 and 20170105936. Examples of anti-cancer agents, drugs, nucleic acid agents, targeting peptides, anti-infective agents, anti-fungal agents, inhibitors, imaging agents, reporter agents, and various other agents that can be used as the cargo molecule in the presently disclosed nanocomposite compositions include, but are not limited to, those described in U.S. Pat. No. 9,616,020 and U.S. Patent Publication Nos. 20180021453, 20170232115, and 20170105936. Examples of methods of binding cargo molecules such as oliognucleotides (e.g., siRNAs) to AuNPs, and linkers used in such conjugates, are shown in U.S. Pat. No. 9,719,089. For example, AuNPs can bind to cargo molecules such as siRNAs by affinity binding to amine groups or can form bonds electrostatically via the replacement of carboxylate in citrate with phosphates in the cargo molecules (e.g., see J. Yue, et. al., Bioconjugate Chem. 2017, 28, 1791-1800 or W-K. Rhim, et. al., Small 2008, 4, 1651-1655).

### EXAMPLES

The embodiments of the present disclosure will be more readily understood by reference to the following examples, which are included merely for purposes of illustration of certain aspects and embodiments of the inventive concepts, and are not intended to be limiting. The following examples and methods describe how to make and use the various formulations and compositions of the present disclosure and are to be construed, as noted above, only as illustrative, and not limitations of the disclosure in any way whatsoever. Those skilled in the art will promptly recognize appropriate variations from the materials and procedures described herein.

### Example 1: Method for the Preparation of Liposomes, ONCs, and OINCs

In a non-limiting embodiment, the liposomal formulations can be made as described in this example. Liposomes (LPs) can be prepared by using a common lipid film hydration method. Briefly, the cationic lipids and fusogenic lipids are separately dissolved in tert-butanol at a concentration of 5 mg/ml. For the preparation of AuNP-siRNA loaded LPs, firstly an AuNP-siRNA conjugate material containing 10 μg AuNPs and 1 ug siRNA (a ratio of 10:1 w/w) is formed by incubating the AuNP and siRNA 15 min at room temperature in 1 ml RNase/DNase-free water. Then, 12.5 μg of each lipid (1:1 w/w) is combined in a glass tube in the presence of excess tert-butanol. During vortexing of the lipids, the conjugate material is added drop by drop onto the lipid mixture, followed by the addition of Tween-20 (1.4 μg) at a ratio of 1:18 w/w of total lipids. The mixture is dried overnight under vacuum conditions in lyophillizer. RNase/DNase-free water (1.0 ml) is added onto the dried film and vortexed for 2 min. This material is then passed through an extruder using polycarbonate membrane (pore size: 0.1 μm) to form the nanoparticles. Non-AuNP-containing siRNA-LPs, and empty-LPs are prepared in the same way, except with only the addition of siRNA for the siRNA-LPs, and water for the empty-LPs in lieu of the AuNP-siRNA conjugate. In non-limiting examples, the AuNP:siRNA ratio in the formulations can be in a range of 1:5 to 1:20 (w/w).

Non-limning examples of lipid formulations used for forming the OINCs are shown in Table 1.

TABLE 1
Lipid compositions used for preparation of select siRNA-ONCs

| Formulation | | Ratio of Lipid | Lipid/siRNA |
|---|---|---|---|
| ID | Lipid Compositions | Type(s) (w/w) | (w/w) ratio |
| E-LPs | DOTAP:DOPE | 50:50 | 25:0 |
| F1 | DOTAP:DOPC | 50:50 | 25:1 |
| F2 | DOTAP:DOPE:DOPC | 40:10:50 | 25:1 |
| F3 | DOTAP:DOPE:DOPC | 30:30:40 | 25:1 |
| F4 | DOTAP:DOPE:DOPC:PE-PEG | 30:10:40:20 | 25:1 |
| F5 | DOTAP:DOPE:DOPC:PE-PEG | 30:20:40:10 | 25:1 |
| F6 | DOTAP:DOPE:DOPC:PE-PEG | 30:25:40:5 | 25:1 |
| F7 | DOTAP:DOPE | 50:50 | 25:1 |
| F8 | DOTAP:DOPE:PE-PEG | 50:50:0.125 mol % | 25:1 |
| F9 | DOTAP:DOPE:PE-PEG | 50:50:0.25 mol % | 25:1 |
| F10 | DOTAP:DOPE:PE-PEG | 50:50:0.5 mol % | 25:1 |

### Example 2

In certain embodiments of the present disclosure, the lipid component comprises a net-positively charged phospholipid (cationic lipid) 1,2-dioleoyl-3-trimethylammonium-propane (DOTAP), a neutral phospholipid (fusogenic lipid) 1,2-Dioleoyl-sn-glycero-3-phosphoethanolamine (DOPE), and a pore-forming surfactant, Tween-20 (polyethylene glycol sorbitan monolaurate). This lip component thus has a net positive charge. This forms the lipid component of both an ONC and an OINC. The OINC is formed with the addition of a negatively charged biocompatible cargo-AuNP conjugate which is substantially encapsulated by the lipid component. As noted elsewhere herein, in non-limiting embodiments, the cargo may be selected from molecules (e.g. siRNA, miRNA, peptides, and proteins) that activate cell death via their respective pathways. In non-limiting embodiments, the genes targeted by the cargo molecules (target genes) may be cystathione-β-synthatase (CBS) or mitochondrial calcium uptake 1 (MICU1) that comprise 18 to 30, 19 to 25, 20 to 23, or 21 contiguous nucleobases or nucleobase pairs, whereas cargo peptides may be in the range of, for example, 20 to 60 amino acids in length. In these embodiments, OINCs can activate cell death that is highly desirable to regress the growth of a cancerous or pre-cancerous or hyperplastic mammalian cell (e.g., a human cell).

### Example 3

In certain embodiments, a neutralization approach is taken in forming the OINCs using a citrate capped 20 nm negatively charged gold nanoparticle (AuNP) as a core conjugate with MICU1 siRNA (CBS siRNA, or other siRNAs could be used as well) into DOTAP/DOPE-based cationic liposomes (LPs), denoted as AuNP-MICU1 siRNA-LPs. The size of AuNP-MICU1 siRNA-LPs is almost 100 nm, having an almost neutral charge (e.g., <10 mV) and an above-95% loading efficiency. As shown in the results discussed elsewhere herein, the AuNP-MICU1 siRNA-LPs exhibited enhanced efficacy in silencing mRNA, requiring 3-4 fold lower siRNA concentrations than MICU1 siRNA-LPs formed without AuNPs, or commercially available transfection reagents, such as Hiperfect. In results shown elsewhere herein, enhanced silencing was reflected in clonal growth assays; AuNP-MICU1 siRNA-LPs inhibited clonal growth of epithelial ovarian cancers (EOCs) more efficiently (˜90%) than MICU1 siRNA-LPs (˜40%) or Hiperfect (˜20%). The AuNP-MICU1 siRNA-LPs inhibited tumor growth more effectively (˜75%) compared to MICU1 siRNA-LPs (˜35%). The inhibition is mediated via notable apoptosis of tumor cells around 11-fold higher than that of MICU1 siRNA-LPs without any toxic effects.

Thus, the present disclosure describes the successful use of AuNP-doped nanoformulations of siRNA in delivering cargo molecules to targets in vivo for inhibiting protein expression, for example, by silencing mRNA translation.

While the present disclosure has been described herein in connection with certain embodiments so that aspects thereof may be more fully understood and appreciated, it is not intended that the present disclosure be limited to these particular embodiments. On the contrary, it is intended that all alternatives, modifications and equivalents are included within the scope of the present disclosure as defined herein. Thus the examples described above, which include particular embodiments, will serve to illustrate the practice of the inventive concepts of the present disclosure, it being understood that the particulars shown are by way of example and for purposes of illustrative discussion of particular embodiments only and are presented in the cause of providing what is believed to be the most useful and readily understood description of procedures as well as of the principles and conceptual aspects of the present disclosure. Changes may be made in the formulation of the various compositions described herein, the methods described herein or in the steps or the sequence of steps of the methods described herein without departing from the spirit and scope of the present disclosure. Further, while various embodiments of the present disclosure have been described in claims herein below, it is not intended that the present disclosure be limited to these particular claims.

### REFERENCES

Torchilin V. P (2005). Recent advances with liposomes as pharmaceutical carriers. Nat Rev Drug Discov. 4(2):145-60.
Elbashir, S. M. et al (2001). Duplexes of 21-nucleotide RNAs mediate RNA interference in cultured mammalian cells. Nature 411, 494-498.
McCaffrey, A. P. et al (2002). Gene expression: RNA interference in adult mice. Nature 418, 38-39.
Kanasty R, Dorkin J R, Vegas A and Anderson D (2013). Delivery materials for siRNA therapeutics. Nat Mater. 12:967-77.
Ernsting M J, Murakami M, Roy A, Li S-D (2013). Factors controlling the pharmacokinetics, biodistribution and intratumoral penetration of nanoparticles. J Control Release. 172:782-94.
Lieleg O, Baumgartel R M, Bausch A R. Selective Filtering of Particles by the Extracellular Matrix: An Electrostatic Bandpass (2009). Biophys J. 97:1569-77.
Miller C R, Bondurant B, McLean S D, McGovern K A, O'Brien D F (1998). Liposome-Cell Interactions in Vitro: Effect of Liposome Surface Charge on the Binding and Endocytosis of Conventional and Sterically Stabilized Liposomes. Biochemistry. 37:12875-83.
Chonn A, Cullis P, Devine D (1991). The role of surface charge in the activation of the classical and alternative pathways of complement by liposomes. J Immunol. 146:4234-41.
Nomura T, Koreeda N, Yamashita F, Takakura Y, Hashida M (1998). Effect of particle size and charge on the disposition of lipid carriers after intratumoral injection into tissue-isolated tumors. Pharm Res. 15:128-32.
Landen C N, Chavez-Reyes A, Bucana C, Schmandt R, Deavers M T, Lopez-Berestein G, et al (2005). Therapeutic EphA2 gene targeting in vivo using neutral liposomal small interfering RNA delivery. Cancer Res. 65:6910-8.
Halder J, Kamat A A, Landen C N Jr. Han L Y, Lutgendorf S K, Lin Y G, et al (2006). Focal adhesion kinase targeting using in vivo short interfering RNA delivery in neutral liposomes for ovarian carcinoma therapy. Clin Cancer Res. 12:4916-24.
Yue, J, Feliciano, T J, Li, W, Lee, A and Teri W. Odom, T W (2017). Gold Nanoparticle Size and Shape Effects on Cellular Uptake and Intracellular Distribution of siRNA Nanoconstructs. Bioconjugate Chem. 2017, 28, 1791-1800.
Rhim, W-K, Kim, J-S and Nam, J-M (2008). Lipid-Gold-Nanoparticle Hybrid-Based Gene Delivery. Small 4:1651-1655.

What is claimed is:

1. An organo-inorganic nanocomposite (OINC), comprising:
a lipid membrane component comprising a cationic lipid, a fusogenic co-lipid, and a pore forming surfactant; and
a cargo-inorganic conjugate component comprising a negatively-charged cargo molecule bound electrostatically or covalently to a negatively-charged biocompatible inorganic nanoparticle wherein the cargo-inorganic component is substantially encapsulated within the lipid membrane component forming the OINC.

2. The OINC of claim 1, wherein at least 90% of the cargo-inorganic conjugate component is encapsulated by the lipid membrane component.

3. The OINC of claim 1, wherein the cationic lipid is 1,2-dioleoyl-3-trimethylammonium-propane (DOTAP), the fusogenic co-lipid is 1,2-dioleoyl-sn-glycero-3-phosphoethanolamine (DOPE), and the pore forming surfactant is polyethylene glycol sorbitan monolaurate.

4. The OINC of claim 1, wherein the cargo molecule is selected from the group consisting of short interfering ribonucleic acids (siRNA), micro RNAs (miRNA), peptides, proteins, and fluorescently labelled dyes.

5. The OINC of claim 1, wherein the negatively-charged biocompatible inorganic nanoparticle is a citrate capped 20 nm gold nanoparticle (GNP or AuNPs).

6. The OINC of claim 1, wherein the cargo molecule is covalently-bound to the negatively-charged biocompatible inorganic nanoparticle.

7. The OINC of claim 1, wherein the cargo molecule is electrostatically-bound to the negatively-charged biocompatible inorganic nanoparticle.

8. The OINC of claim 1, disposed in a pharmaceutically acceptable carrier.

9. The OINC of claim 1, wherein the cargo-inorganic conjugate component comprises organic cargo molecules and biocompatible inorganic nanoparticles in a ratio of about 1:5 (w/w) to about 1:20 (w/w).

10. The OINC of claim 1, wherein the cargo molecule is an siRNA and the OINC has at least a 90% knock-down rate of the target gene with a nanomolar dose of the siRNA, wherein the nanomolar dose is in a range of about 25 nM to about 100 nM.

11. The OINC of claim 1, comprising an average diameter in a range of about 50 nm to about 300 nm.

12. The OINC of claim 1, comprising a charge less than about 10 mV.

13. The OINC of claim 1, comprising an ability to take up via caveolae-mediated endocytosis or macropinocytosis and to deliver the cargo molecule into cell cytosol via escape from endosomes and/or lysosomes

14. The OINC of claim 1, comprising activity against the proliferation and clonal expansion of cancer cells.

15. The OINC of claim 1, wherein the cargo molecule is selected from mitochondrial calcium uptake 1 (MICU1) siRNA and cystathione-β-synthatase (CBS) siRNA.

16. The OINC of claim 1, wherein the cargo molecule is an siRNA that inhibits translation of a oncogene involved in cancer.

17. A method of treating a cancer in a subject, comprising administering a therapeutically-effective amount of the OINC of claim 1 to a subject in need of such treatment.

18. The method of claim 17, wherein the cancer is an ovarian cancer.
